(19) United States
(12) Patent Application Publication
(10) Pub. No.: US 2011/0077733 A1
(43) Pub. Date: Mar. 31, 2011

(54) LEAFLET CONTACTING APPARATUS AND METHOD

(75) Inventor: Jan Otto Solem, Bjarred (SE)

(73) Assignee: EDWARDS LIFESCIENCES CORPORATION, Irvine, CA (US)

(21) Appl. No.: 12/567,047

(22) Filed: Sep. 25, 2009

(51) Int. Cl. A61F 2/24; A61M 29/00

(52) U.S. Cl. 623/2.12; 606/194

(57) ABSTRACT

A prosthesis can include a leaflet contacting member and an anchoring member. The leaflet contacting member can have an outer surface and an aperture that extends through at least a portion of the leaflet contacting member. The outer surface of the leaflet contacting member can be configured to contact one or more leaflets of the heart valve during coaptation of the leaflets, and the contact of the leaflets with the outer surface can prevent the leaflets from contacting one or more wires that extend through the aperture of the leaflet contacting member.

### FIELD

The present disclosure is directed to apparatuses and methods that can be used with various devices that treat defective heart valves and as diagnostic tools for use in connection with the treatment of defective heart valves.

### BACKGROUND

Heart valve disease is a serious problem that involves the malfunction of one or more valves of the heart. The malfunction can manifest itself in a variety of manners. Valve insufficiency, for example, is the failure of a valve to close properly to prevent leaking, or backflow, of blood through the valve. As a result of this leakage, blood is unable to properly flow through the heart.

For example, the normal operation of the mitral valve can be impaired when the mitral valve leaflets fail to coapt or fully close, allowing regurgitated blood to flow from the left ventricle back into the left atrium. Similarly, the normal operation of the tricuspid valve can be impaired when the tricuspid valve leaflets fail to coapt or fully close, allowing regurgitated blood to flow from the right ventricle back into the right atrium.

### SUMMARY

In one embodiment, a prosthesis that can be positioned within an annulus of a heart valve is provided. The prosthesis includes a leaflet contacting member and an anchoring member. The leaflet contacting member can have an outer surface, an inflow side, an outflow side, and an aperture that extends through at least a portion of the leaflet contacting member. The aperture can be sized to allow one or more wires to extend through the leaflet contacting member from the inflow side to the outflow side. The anchoring member can be coupled to the leaflet contacting member and configured to anchor the leaflet contacting member within the annulus of a heart valve. The outer surface of the leaflet contacting member can be configured to contact one or more leaflets of the heart valve during coaptation of the leaflets, and the contact of the leaflets with the outer surface can prevent the leaflets from contacting the one or more wires when positioned to extend through the aperture of the leaflet contacting member.

In specific implementations, the aperture can have a width that is substantially the same as the width of the wire(s) that extend through the aperture, thereby substantially restricting blood flow through the aperture when the wire(s) extend through the aperture. In another specific implementation, a plug member can be configured to restrict blood flow through the aperture when the wire(s) extend through the aperture.

In other specific implementations, the anchoring member can have a compressed state sized to fit within a delivery catheter and an expanded state sized for fixation on at least a portion of a wall of a left atrium. In certain implementations, the anchoring member can also comprise a plurality of loops formed of a shape memory material. In other implementations, the anchoring structure can extend into the right ventricle and be secured to a wall of the right ventricle. In other implementations, the anchoring structure can include an electrode tip coupled to a distal end of the one or more wires.

In other specific implementations, the leaflet contacting member can comprise an expandable member. The expandable member can have an expanded state that restricts blood flow between the leaflets and the leaflet contacting member and a contracted state that allows blood to flow between the leaflets and the leaflet contacting member.

In other specific implementations, the leaflet contacting member can have a length that is substantially equal to a length of a commissure of the leaflets. In yet other specific implementations, the aperture can be positioned at a substantially central location along the length of the leaflet contacting member. Alternatively, the aperture can be positioned off center relative to the length of the leaflet contacting member.

In another embodiment, a system for preventing contact between a wire and one or more valve leaflets is provided. The system includes a prosthetic device and at least one wire. The prosthetic device has a main body that is sized for placement at least partially between two valve leaflets that are movable between an open state and a closed state. The main body includes an opening that extends from a first side of the main body to a second side of the main body. The wire is configured to extend through the opening of the main body from the first side to the second side. The opening is spaced apart from an outer perimeter of the main body so that, when the prosthetic device is placed between the two valve leaflets, the wire does not contact either of the two valve leaflets when the valve leaflets are in the open or closed states.

In specific implementations, the prosthetic device includes an anchoring member that is configured to securely hold the main body at least partially between the two valve leaflets. In other specific implementations, the anchoring member has a compressed state sized to fit within a delivery catheter and an expanded state sized for fixation on at least a portion of a wall of a left atrium. The anchoring member can be a plurality of loops formed of a shape memory material.

In other implementations, the opening is substantially the same size as the one or more wires that extend through the opening, thereby substantially restricting blood flow through the opening when the one or more wires extend through the opening. In other implementations, the main body comprises an expandable member having an outer surface that surrounds the aperture. The expandable member has an expanded state that restricts blood flow between the leaflets and the outer surface of the expandable member and a contracted state that allows blood to flow between the leaflets and the outer surface of the leaflet contacting member. In yet other implementations, the main body has a length that is substantially equal to a length of a commissure of the leaflets.

In another embodiment, a method is provided for delivering a wire through a valve of the heart. The method comprises providing a leaflet contacting member having an outer surface and an aperture passing through the leaflet contacting member; positioning the leaflet contacting member at least partially between two leaflets of a valve so that the outer surface of the leaflet contacting member contacts the leaflets when the leaflets undergo coaptation; and passing one or more wires through the aperture of the leaflet contact member. The aperture is positioned such that the one or more wires do not contact the leaflets.

In specific implementations, the method includes providing an anchoring member coupled to the prosthetic device and anchoring the leaflet contacting member at least partially between the two leaflets. In yet other implementations, the method comprises providing a plug member and positioning the plug member at or around the aperture to restrict blood flow through the aperture.

In another embodiment, a diagnostic tool is provided. The tool comprises an elongate member having a proximal portion, a distal portion, and a distal opening. The elongate member has a lumen that extends from the proximal portion to the distal portion. The distal opening is in fluid connection with the lumen. The tool also comprises a temporary coaptation member coupled to the distal portion of the elongate member at an attachment portion. The temporary coaptation member is configured to be positioned between two leaflets. The attachment portion is located proximally to the distal opening in the elongate member.

In specific implementations, a fluid delivery device is coupled to the proximal portion of the diagnostic tool and configured to pump fluid from the proximal portion to the distal portion. In another specific implementation, the temporary coaptation member can comprise an expandable member that has an expanded state that restricts blood flow between the leaflets and the expandable member and a contracted state that allows blood to flow between the leaflets and the expandable member.

In another embodiment, a method of using a diagnostic tool for determining an effectiveness of a temporary coaptation member to minimize regurgitation in a heart valve in a body of a patient is provided. The leaflets are movable between a closed state and a open state. The method comprises providing an elongate member having a proximal portion and a distal portion; providing a temporary coaptation member coupled to the distal portion of the elongate member; positioning the temporary coaptation member at least partially between two leaflets of heart valve; monitoring the effectiveness of the temporary coaptation member in restricting blood flow between the leaflets and the temporary coaptation member when the leaflets are in the closed state; and removing the temporary coaptation member from the patient's body.

In specific implementations, the method further comprises providing a lumen that extends through the elongate member from the proximal portion to the distal portion, the lumen being in fluid connection with a distal opening in the elongate member; positioning the distal opening of the elongate member in the left ventricle; and delivering fluid through the lumen into the left ventricle to increase a fluid pressure in the left ventricle.

In specific implementations, the method comprises moving the temporary coaptation member to a different position at least partially between the leaflets; and monitoring the effectiveness of the temporary coaptation member in restricting blood flow between the leaflets and the temporary coaptation member when the leaflets are in a closed state.

In specific implementations, the temporary coaptation member comprises an expandable member that has an expanded state that restricts blood flow between the leaflets and the expandable member and a contracted state that allows blood to flow between the leaflets and the expandable member.

The foregoing and other objects, features, and advantages of the embodiments disclosed herein will become more apparent from the following detailed description, which proceeds with reference to the accompanying figures.

### BRIEF DESCRIPTION OF THE DRAWINGS

FIG. 1 illustrates a schematic view of a partial cross-section of the heart indicating its general anatomy.

FIG. 2 illustrates a schematic view of an implanted medical device having an electrical lead extending into the heart of a patient.

FIG. 3 illustrates a schematic view of a tricuspid valve in a closed position.

FIG. 4 illustrates a schematic view of a tricuspid valve in a closed position with a lead or wire interfering with the coaptation of the leaflets.

FIG. 5 illustrates a schematic view of partial cross-section of a heart showing a leaflet contacting member with a lead or wire extending through an aperture in the leaflet contacting member, according to one embodiment.

FIG. 6 illustrates a schematic view of a tricuspid valve in a closed position showing a leaflet contacting member with a lead or wire extending through an aperture in the leaflet contacting member.

FIG. 7 illustrates a leaflet contacting member with an anchoring portion.

FIG. 8 illustrates a leaflet contacting member with an anchoring portion.

FIG. 9 illustrates a portion of a leaflet contacting member.

FIG. 10A illustrates a front view of leaflet contacting member with a lead or wire passing through an aperture in the leaflet contacting member.

FIG. 10B illustrates a perspective view of leaflet contacting member with a lead or wire passing through an aperture in the leaflet contacting member.

FIG. 10C illustrates a side view of leaflet contacting member with a lead or wire passing through an aperture in the leaflet contacting member, with the leaflet contacting member in an expanded configuration.

FIG. 10D illustrates a side view of leaflet contacting member with a lead or wire passing through an aperture in the leaflet contacting member, with the leaflet contacting member in a collapsed configuration.

FIG. 11A illustrates a bottom view of a valve having a leaflet contacting member positioned within the annulus, shown with the leaflet contacting member in a collapsed position.

FIG. 11B illustrates a bottom view of a valve having a leaflet contacting member positioned within the annulus, shown with the leaflet contacting member in an expanded position

FIG. 12A illustrates a schematic view of a partial cross-section of a heart, shown with a leaflet contacting member in a partially collapsed configuration and with a lead or wire passing through an aperture in the leaflet contacting member.

FIG. 12B illustrates a schematic view of a partial cross-section of a heart, shown with a leaflet contacting member in an expanded configuration and with a lead or wire passing through an aperture in the leaflet contacting member.

FIG. 13A illustrates an embodiment of a leaflet contacting member that has an anchoring portion.

FIG. 13B illustrates an embodiment of a leaflet contacting member that has an anchoring portion.

FIG. 13C illustrates an embodiment of a leaflet contacting member that has an anchoring portion.

FIG. 13D illustrates an embodiment of a leaflet contacting member that has an anchoring portion.

FIG. 14 illustrates an embodiment of a leaflet contacting member that has an anchoring portion and non-expandable aperture-containing portion.

FIG. 15 illustrates a view of a leaflet contacting member that has an anchoring portion and non-expandable aperture-containing portion, with the leaflet contacting member positioned within an annulus of a valve.

FIG. 16 illustrates a diagnostic tool for testing the potential effectiveness of a leaflet contacting member.

FIG. 17 illustrates a schematic view of a cross-section of a heart, showing the diagnostic tool of FIG. 16 positioned within the heart.

### DETAILED DESCRIPTION

The following description is exemplary in nature and is not intended to limit the scope, applicability, or configuration of the invention in any way. Various changes to the described embodiment may be made in the function and arrangement of the elements described herein without departing from the scope of the invention.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

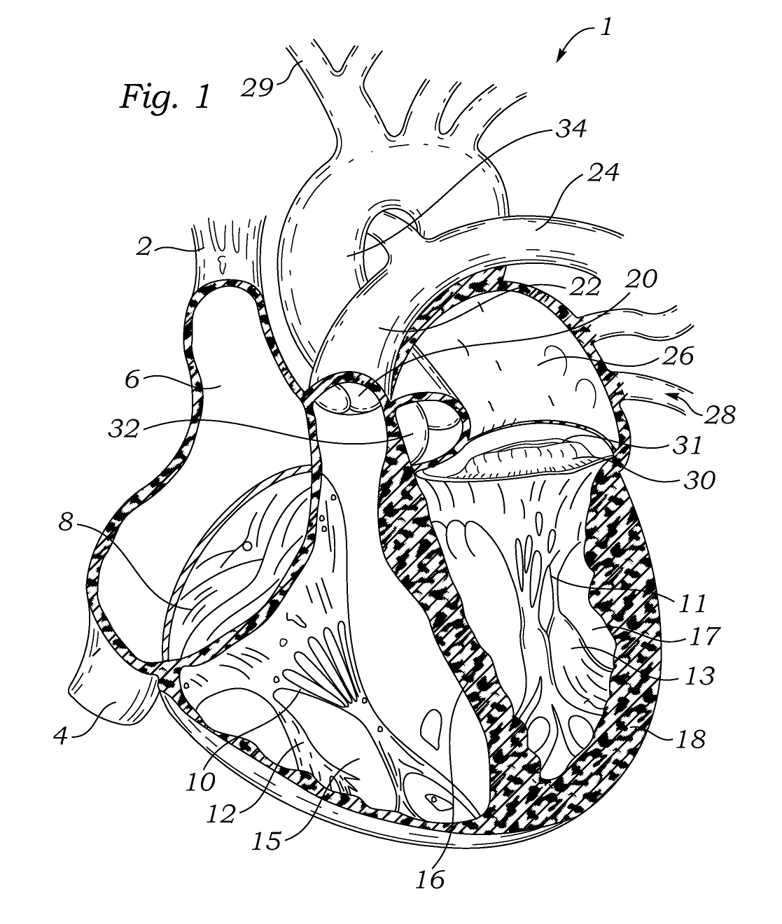

Referring to FIG. 1, the general anatomy of a heart 1 will be described. Blood passes through the superior vena cava 2 and the inferior vena cava 4 into the right atrium 6 of the heart 1. The tricuspid valve 8 controls blood flow between the right atrium 6 and the right ventricle 15. To prevent regurgitation of blood back into the right atrium, the tricuspid valve 8 is substantially closed when blood is pumped out from the right ventricle 15 to the lungs. During this period, more blood enters the right atrium 6. Thereafter, the tricuspid valve 8 opens to fill the right ventricle 15 again with the blood from the right atrium 6. Free edges of leaflets of the tricuspid valve 8 are connected via chordae tendinae 10 to papillary muscles 12 for controlling the opening and closing of the tricuspid valve 8.

The blood that leaves the right ventricle 15 is pumped through the pulmonary valve 20 to the pulmonary artery 22, which branches into arteries leading to each lung. Blood from the lungs is then pumped through pulmonary veins 28 into the left atrium 26 of the heart 1. The mitral valve 30 opens and closes to control blood flow between the left atrium 26 and the left ventricle 17. To prevent regurgitation of blood back into the left atrium, the mitral valve 30 is substantially closed when blood is pumped out from the left ventricle 17 through the aortic valve 32 and into the aorta 34 which branches into arteries leading to all parts of the body. During this period, more blood enters the left atrium 26. Thereafter, the mitral valve 30 opens to fill the left ventricle 17 again with the blood from the left atrium 26. Free edges of leaflets of the mitral valve 30 are connected via chordae tendinae 11 to papillary muscles 13 for controlling the movements of the mitral valve 30.

The function of the heart 1 can be impaired when any of the heart valves do not function properly. For example, one or more heart valves may lose its ability to close properly due to dilation of an annulus around the valve or a prolapsing leaflet. Leaflets can also shrink or be malformed due to disease (e.g., rheumatic disease), and thereby leave a gap in the valve between the leaflets. The inability of the heart valve to fully close can cause blood to leak backwards through the valve. This backwards leaking or regurgitation can impair the function of the heart 1 since more blood will have to be pumped through a regurgitating valve.

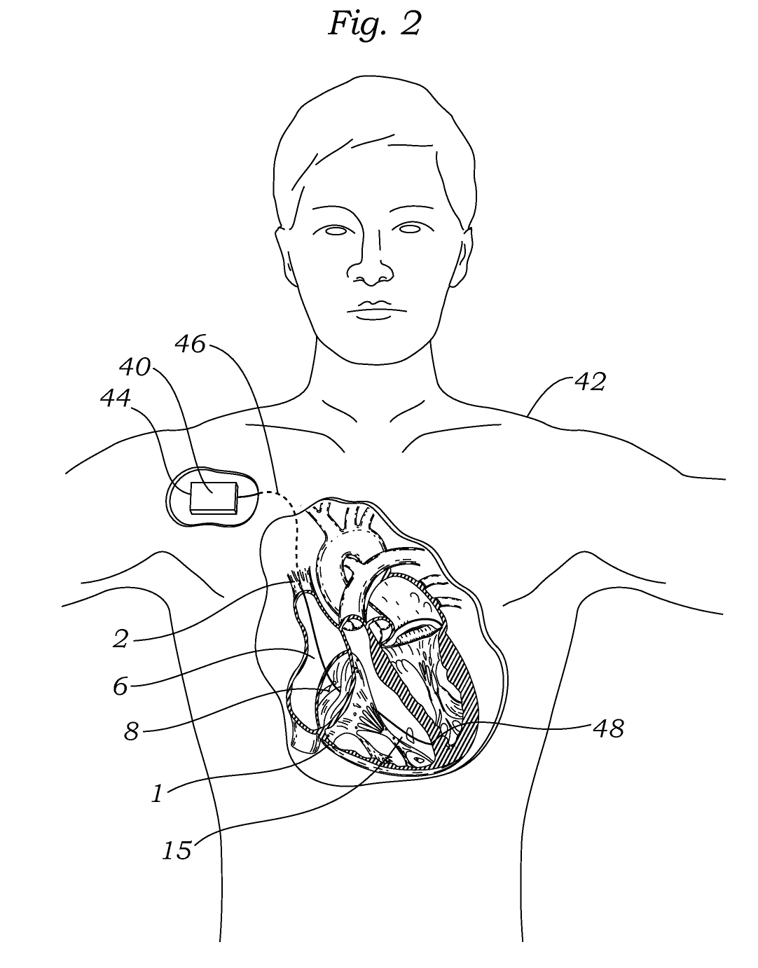

In addition to disease, heart valve function can be adversely affected when the leaflets or annulus of a heart valve come into contact with artificial structures implanted in the body. For example, various illnesses or diseases of the heart can be treated by electrical stimulation, which requires passing one or more leads or wires through a heart valve. FIG. 2, for example, illustrates a schematic view of a body into which an implantable medical device (IMD) has been positioned inside the body of a patient 42. In this embodiment, the IMD comprises a pacemaker 40 that includes a pacemaker generator 44 and at least one pacemaker wire or lead 46 connected to the generator 44. Pacemaker generator 44 generally includes a power source (e.g., a battery) and the necessary wiring and circuitry to generate and deliver electrical impulses to the heart 1 through lead 46.

As shown in FIG. 2, one end of lead 46 is attached to generator 44 and the other end of lead 46 is attached to the heart 1 in order to deliver a desired amount of electrical stimulation to the heart 1. FIG. 2 illustrates an exemplary embodiment where lead 46 passes through the superior vena cava 2, through the right atrium 6, through the tricuspid valve 8, and into the right ventricle 15. A lead tip 48 of the lead 46 can be attached to the heart 1 along a wall of the right ventricle 15.

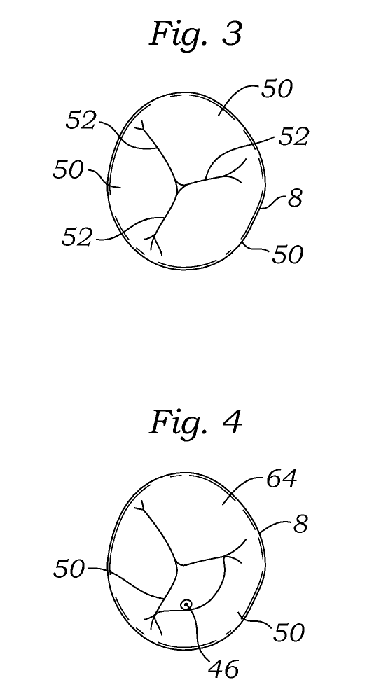

As discussed above, a properly functioning tricuspid valve 8 should be able to close to restrict blood from being regurgitated from the right ventricle back into the right atrium during systole. FIG. 3 illustrates a bottom schematic view of a functioning tricuspid valve 8 in a closed position (shown with the chordae tendinae and other such details removed for clarity). Assuming the tricuspid valve 8 is healthy and not otherwise impeded, adjacent leaflets 50 are able to contact one another along coaptation surfaces 52, as shown in FIG. 3, thereby substantially preventing leakage between the right ventricle and right atrium.

However, as shown in FIG. 4, when one or more wires or leads 46 (or other similar structure) are positioned at or pass through the tricuspid valve annulus, the leaflets 50 of the tricuspid valve 8 can be prevented or hindered from closing properly. For example, direct contact between lead 46 and one or more of the leaflets 50 can result in damage to and/or dislocation of the leaflets 50. Also, over time, repeated contact between lead 46 and leaflets 50 can further damage the leaflets 50, resulting in permanent or chronic valve insufficiency.

The embodiments disclosed herein prevent or substantially restrict contact between the leaflets and one or more leads or wires that pass through the annulus of valve, such as the tricuspid or mitral valves discussed below. Although the embodiments herein are directed to restricting contact between a lead or wire and leaflets, it should be understood that other artificial members or prosthetic devices that have portions that pass through a heart valve can also be prevented from contacting leaflets using the various structures disclosed herein.

To eliminate or minimize contact between the leaflets and leads (or other prosthetic devices that have a portion or element that extends through a heart valve), a leaflet contacting member can be positioned between the leaflets and the leads. The leaflet contacting member is preferably anchored within the annulus and includes one or more apertures through which the one or more leads or wires can pass.

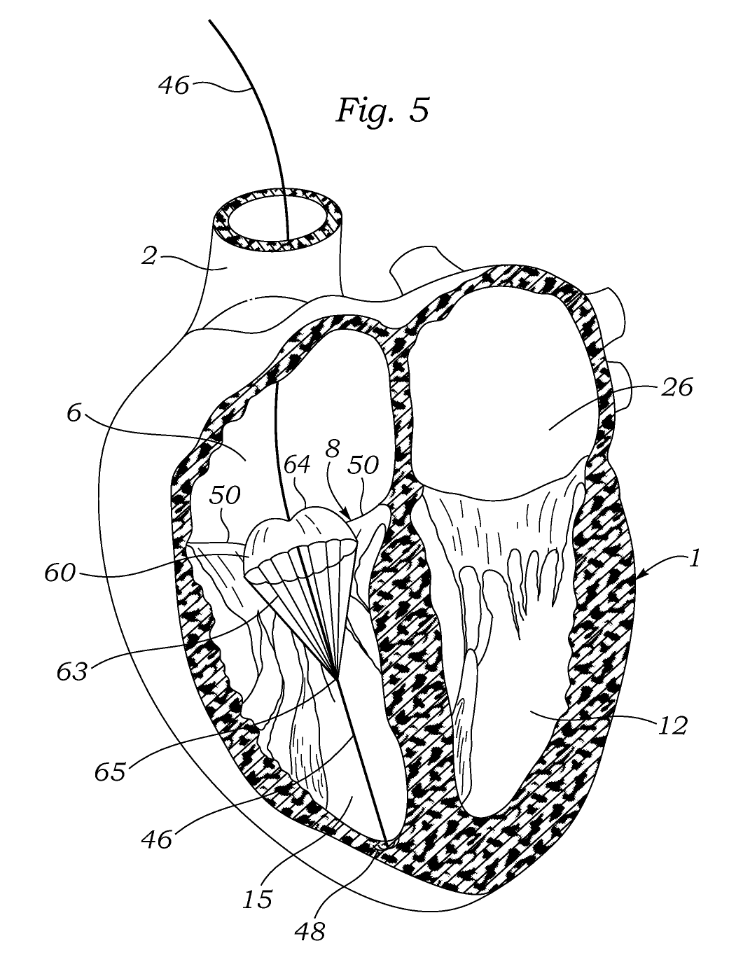

FIG. 5 illustrates a prosthesis device that comprises a leaflet contacting member 60 positioned between leaflets 50 of a valve. Leaflet contacting members as described herein can function as coaptation members when sized and positioned in such a manner to restrict regurgitation. As described in certain embodiments herein, leaflet contacting members can also have expandable portions that expand to further restrict regurgitation.

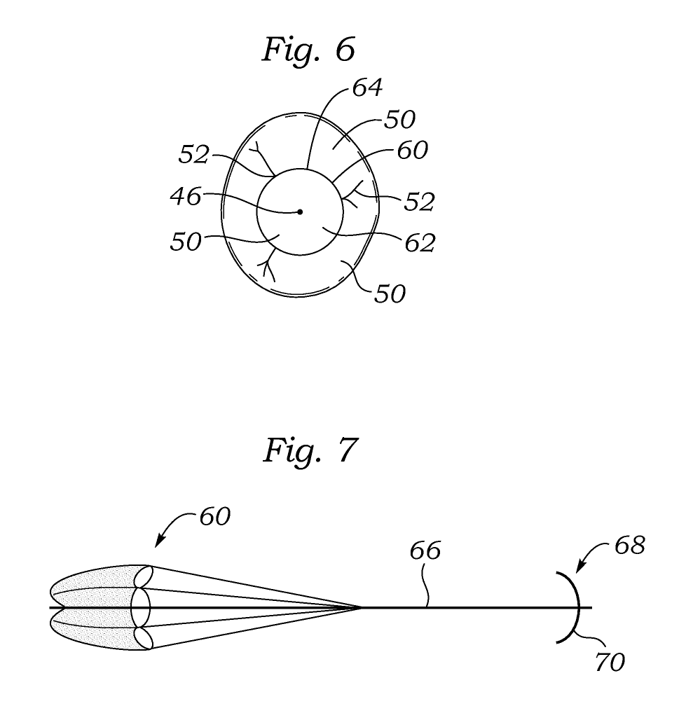

As best seen in FIG. 6, leaflet contacting member 60 can have an outer surface 64 and an aperture or opening 62 that extends through leaflet contacting member 60 from a first side (e.g., top portion) to a second side (e.g., bottom portion). Preferably, aperture 62 extends through at least the portion of leaflet contacting member 60 that contacts leaflets 50. Thus, when leaflet contacting member 60 is positioned at and/or within the valve annulus, lead 46 can extend through aperture 62 without contacting leaflets 50 as the leaflets move between an open and a closed (coapted) state.

As discussed in more detail above, leaflets 50 of an operational tricuspid valve move between an open position during diastole and a closed position during systole. As shown in FIG. 6, when leaflet contacting member 60 is positioned at or within the valve annulus, outer surface 64 of leaflet contacting member 60 will contact one or more leaflets 50 of the tricuspid valve 8 when the leaflets 50 are in the closed position. Thus, outer surface 64 of leaflet contacting member 60 substantially restricts leaflets 50 from contacting lead 46 as the leaflets 50 move between the open and closed positions.

In one embodiment, leaflet contacting member 60 can be anchored to the heart by coupling or securing a portion of leaflet contacting member 60 to lead 46, which is in turn anchored to the heart via a lead tip 48 that is secured to the heart and configured to provide electrical stimulation to the heart. Thus, the electrode tip can provide the necessary anchoring force as well as electrical stimulation to the heart muscle, and the lead 46 can provide a structure for attaching a portion of the leaflet contacting member 60.

For example, leaflet contacting member 60 can have a plurality of enforcement members 63 that extend from a portion of the leaflet contacting member 60 and are secured to the lead 46 at one or more attachment locations 65. The enforcement members 63 can stabilize the shape of the leaflet contacting member 60 in the closed position. The enforcement members 63 can be an integrated part of the leaflet contacting member 60 or they can be attached to the leaflet contacting member 60 by, for example, gluing or tying. The enforcement members 63 can also prevent leaflet contacting member 60 from over extending and turning over. Enforcement members 63 can be attached to leaflet contacting member 60 at various locations; however, as shown in FIG. 5, they are preferably coupled at or near an outer perimeter of the leaflet contacting member 60 to provide further structural support to the device.

Although the above embodiment describes leaflet contacting member 60 as being anchored to the heart by coupling it to lead 46 (with lead tip 46 anchoring lead 46 to the heart), leaflet contacting member 60 can be alternatively, or additionally, anchored at one or more portions of the heart 1 to substantially hold leaflet contacting member 60 in position at and/or within the valve annulus. Leaflet contacting member 60 can be anchored in a number of different ways. U.S. Patent Publication Nos. 2006/0241745 and 2006/0058871, both of which are incorporated by reference herein in their entirety, disclose various anchoring mechanisms and leaflet contacting members that can be used in connection with the embodiments disclosed herein.

For example, referring to FIG. 7, leaflet contacting member 60 can be configured to be coupled to a supporting structure that includes one or more connecting members 66 and an anchoring member 68. Connecting members 66 can be configured to be coupled to leaflet contacting member 60 at a first portion and to an anchoring member 68 at a second portion.

In the embodiment shown in FIG. 7, a leaflet contacting portion of leaflet contacting member 60 is coupled to connecting member 66. Connecting member 66 is also coupled to anchoring member 68, which is configured to be secured to a portion of the heart. For example, anchoring member 68 can be a disk-shaped element 70 that is arranged to contact a heart wall portion, such as a ventricular wall or interventricular septum. A portion of the connecting member 66 can extend through the heart wall and the disk-shaped element 70 to substantially prevent the anchoring member 68 from migrating through the heart wall. The anchoring member 68 can further comprise a hook, barb, or the like for engaging the wall. The disk-shaped element 70 can be compressed for insertion through the heart wall and can assume its disk-shape when a compressing force is released. Using the structure shown in FIG. 7, leaflet contacting member 60 can be coupled to a wall of the ventricle independently (separately) from the electrode tip 48.

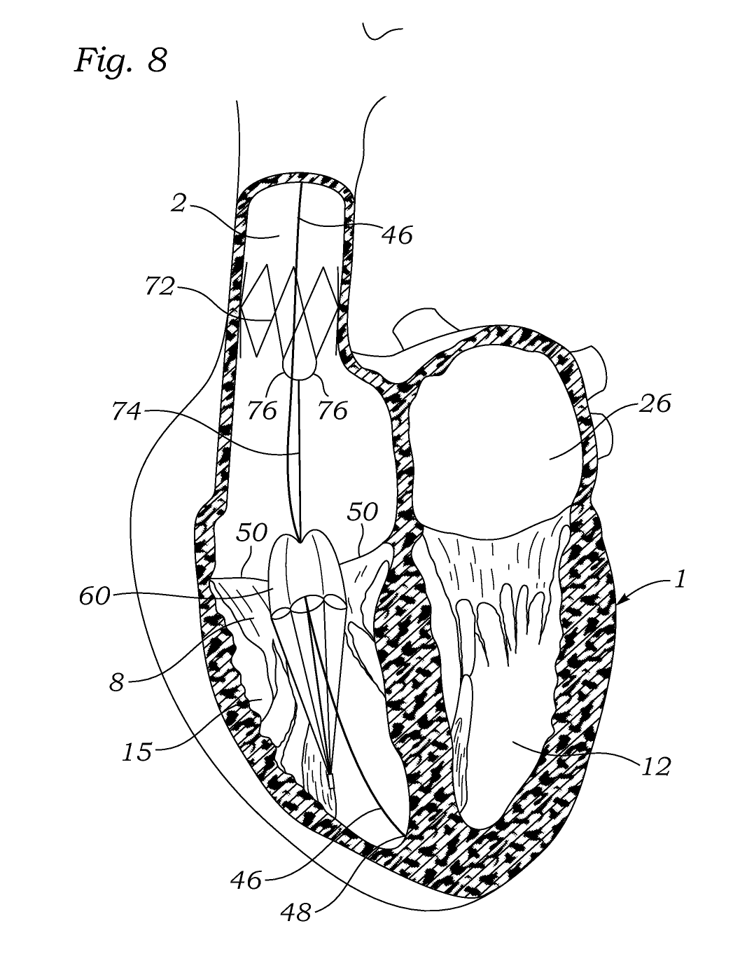

Alternatively, or in addition to a ventricular anchor, leaflet contacting member 60 can be anchored above the tricuspid valve in the right atrium or the superior vena cava. For example, as shown in FIG. 8, the position of the leaflet contacting member 60 can be fixed using an anchoring member 72 that is placed in the superior vena cava 2 for engaging the wall of the vessel. A connecting member 74 can extend through the right atrium 6 between the superior vena cava 2 and the tricuspid valve 8 for connecting leaflet contacting member 60 to anchoring member 72. Anchoring member 72 can comprise, for example, an expandable stent structure that expands radially to contact the internal walls of the superior vena cava 2. As shown in FIG. 8, the connecting member 74 can alternatively branch into two arms 76 which are attached to struts of the anchoring member 72.

FIG. 8 illustrates another embodiment of a leaflet contacting member 60 that has a wire 46 extending through leaflet contacting member 60 and having a lead tip 48 coupled to the heart. Wire 46 can extend separately from the connecting member 74 (as shown in FIG. 8). Alternatively, wire 46 can extend alongside and/or be coupled to connecting member 74 and/or other portions of the anchoring members.

Leaflet contacting member 60 can be configured in a variety of shapes and sizes, so long as it functions to prevent or limit direct contact between one or more leads or wires and the leaflets and/or annulus of a heart valve. In addition to preventing the lead or wire from contacting leaflets and impeding the functioning of the leaflets, the leaflet contacting member is preferably configured and/or positioned within the annulus such that it does not adversely affect the normal function of a non-diseased or non-impaired valve. To the extent the valve suffers from regurgitation, the leaflet contacting member can restrict the leakage of blood between the valve leaflets by at least partially blocking gaps between leaflets when the valve is closed. As discussed above, when valve leaflets fail to properly close, heart valves can suffer from leaks or regurgitation. By selectively positioning the leaflet contacting member 60 within a valve annulus, the leaflet contacting member can function to at least partially block gaps that are present between valve leaflets.

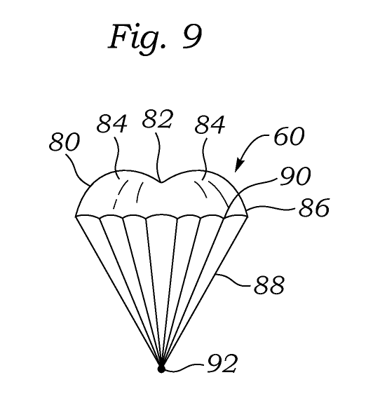

In one embodiment, leaflet contacting member can include a radially collapsible member with an aperture through which the lead(s) or wire(s) can be passed. Referring to FIG. 9, leaflet contacting member 60 can comprise one or more flaps 80 that surround the aperture that the lead passes through (not shown in FIG. 9) in a substantially symmetrical manner. Flaps 80 can be attached to a centrally located attachment point 82, which defines the aperture of the leaflet contacting member.

In a preferred embodiment, a substantially fluid-tight seal is established between the lead and the leaflet contacting member to restrict leakage of blood through the aperture of the leaflet contacting member. Thus, in certain implementations, the aperture or opening can have a size and shape (e.g., width) that is substantially the same as the collective width of the one or more wires that extend through the aperture. Thus, the seal can be formed by sizing the aperture to form a tight fit around the lead. Alternatively, or in addition to using relatively tight tolerances, a plug member in the form of an additional structural member and/or sealing material can be coupled and/or adhered to leaflet contacting member 60 or the lead 46 to substantially seal any gaps between the wire(s) and the aperture.

Flaps 80 can be hinged at the attachment point 82 so that they are moveable between an open position where the flaps 80 are closer together in a radially collapsed state, and a closed position (FIG. 9) where the flaps extend in a radially expanded state. The flaps 80 have contact surfaces 84 which face the forward flow of blood in the native heart valve or the vessel and which are arranged to contact the leaflets of the native heart valve or the vessel wall in the closed position.

The contact surfaces 84 can be configured so that they come into contact with the leaflets of the native heart valve or the vessel wall before the flaps 80 extend to a fully radially extended state. In this manner, the flaps 80 can contact the leaflets of the native heart valve or the vessel wall in a coaptation area 86 of the contact surfaces 84. By oversizing the flaps 80 so that they can expand to a greater diameter than required, leaflet contacting member 60 can be positioned within the annulus with less precision and still function effectively. Thus, leaflet contacting member 60 may not need to be precisely centrally positioned in the native heart valve or the vessel. In fact, it may be desirable to position the leaflet contacting member in a non-central position if leaks are occurring in the valve at positions other than the center.

If necessary, contact surface 84 can be strengthened by enforcement members (e.g., strings or wires) 88 that extend from a rim 90 to a fixation point 92. Fixation point 92 can be coupled to a connecting member 66 of the type shown in FIG. 7, or to lead 46 if lead 46 itself serves as the connecting member (as discussed above). The enforcement members 88 can stabilize the shape of the flaps 80 in the closed position. The enforcement members 88 can be an integrated part of the flaps 80 or they may be attached to the flaps 80 by, for example, gluing or tying. The enforcement members 88 can also prevent the flaps 88 from over extending and turning over.

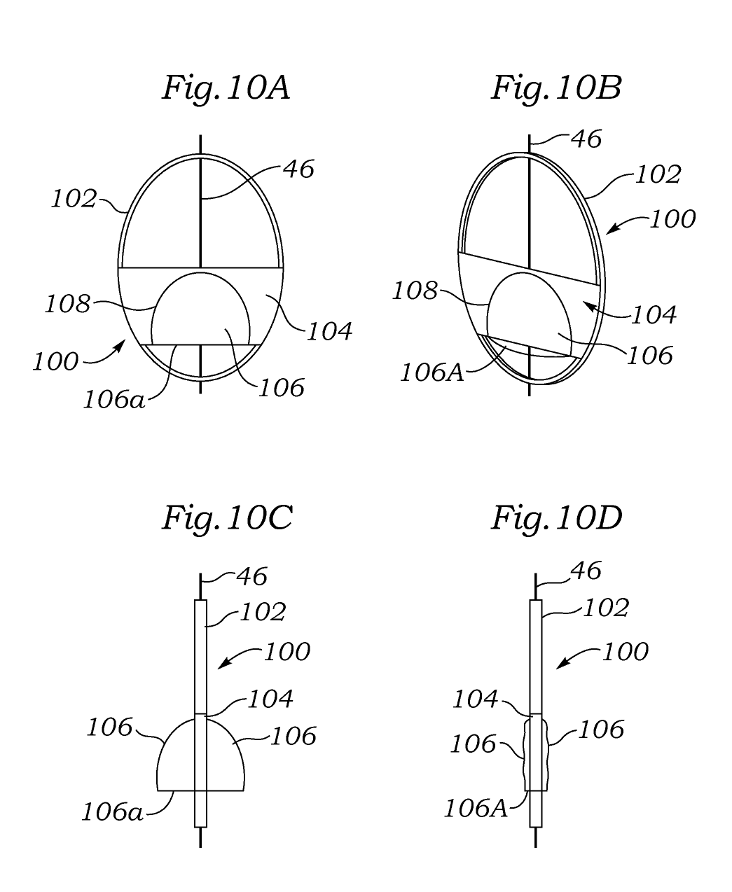

Other structures that surround a lead or wire to prevent contact between the lead and leaflets of a heart valve can be used. For example, FIGS. 10A-10D (discussed below) illustrate another leaflet contacting member with a radially collapsible portion. In addition, the structures disclosed herein can be used in connection with valves other than the tricuspid valve. For example, the leaflet contacting member illustrated in FIGS. 10A-10D is described below for use in connection with a mitral valve.

Referring to FIGS. 10A-10D, leaflet contacting member 100 includes a pocket 106 formed from flexible material 104 disposed on a ring 102. As best seen in FIG. 10B, the pocket 106 includes a lower open end 106A that, when properly oriented within a mitral valve 120 of a heart 124, expands from pressure in the left ventricle as the mitral valve 120 closes, blocking any openings between the mitral valve leaflets 122. Further, the pocket 106 contracts or deflates as the mitral valve 120 opens, maximizing blood flow from a left atrium 126 to a left ventricle 128.

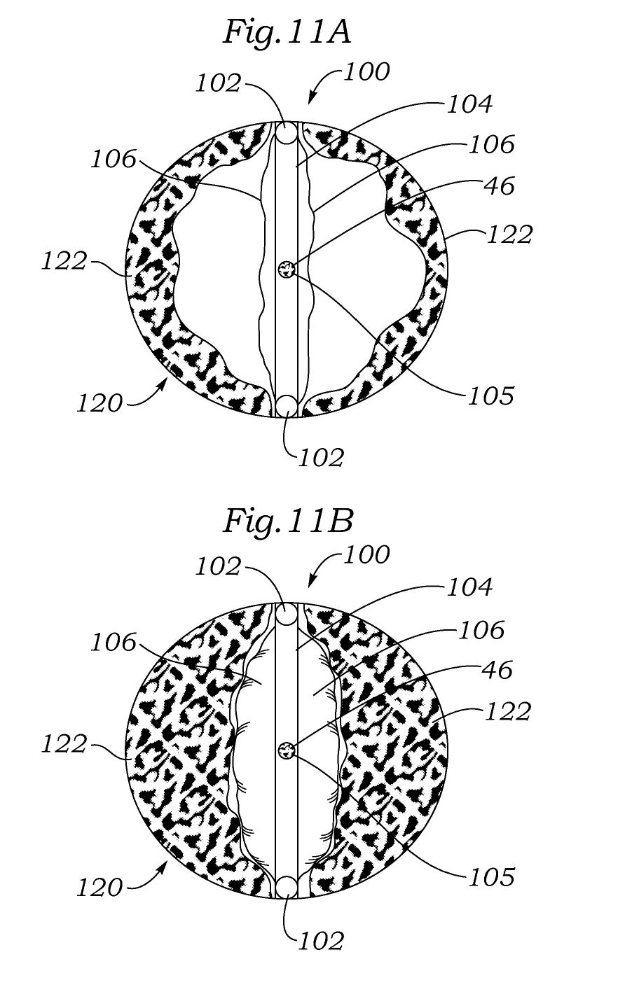

Pocket 106 comprises at least one aperture through which one or more leads 46 can pass. Preferably, as discussed above, lead 46 passes through the aperture in a substantially fluid-tight manner to prevent leakage between lead 46 and pocket 106. Referring to FIGS. 11A and 11B, an aperture 105 can be configured to pass through the pocket 106 of the leaflet contacting member 100. FIG. 11A shows leaflets 122 in an open position and the pocket 106 in a collapsed position to permit blood to flow through the mitral valve 120. FIG. 11B shows leaflets 122 in a closed position and the pocket 106 in an expanded position, with the leaflets 122 abutting an outside surface of the pocket 106. In both the open and closed positions (FIGS. 11A and 11B), an outside surface of the leaflet contact member 100 is positioned between the wire 46 and the leaflets 122. Thus, the wire 46 is restricted from contacting the leaflets 122 and disrupting or interfering with the operation of the leaflets 122. Although shown in a substantially central position in FIGS. 11A and 11B, it should be understood that aperture 105 can be located elsewhere relative to the leaflet contacting member, including in an off-center position.

Pocket 106 can be created by gluing, stitching, or otherwise adhering at least two layers of the flexible material 104 at or around line 108. These layers can be achieved with two distinct pieces of material, or a single piece of material folded against itself. Preferably, the flexible material 104 is made from pericardial tissue or other biological or artificial materials with similar flexibilities, such as bovine tissue, polyurethane, or as described in U.S. Pat. No. 6,764,510, the contents of which are herein incorporated by reference. The shape of the pocket 106 and the flexibility of the flexible fabric 108 allow the pocket 106 to achieve a deflated position, as best seen in FIGS. 10D, 11A, and 12A and an expanded position as best seen in FIGS. 10C, 11B and 12B.

While the pocket 106 can be shaped in a variety of different configurations, pocket shapes that facilitate entry and escape of blood from the pocket 106, such as the rounded arch-shape of pocket 106, are preferred. Configurations of the pocket 106 that include sharp corners or rough seams are less preferred due to their disruptive effect on blood flow into and out of the pocket 106. Preferably, the pocket 106 also includes an overall length similar to that of the mitral valve 120 and more preferably substantially the length of the mitral valve commissure, allowing the pocket 106 to fill any openings that may be present along the length of leaflets 122, as seen best in FIG. 11B. While a single pocket 106 is preferred, additional pockets or partitions within the pocket can also be included in the present invention.

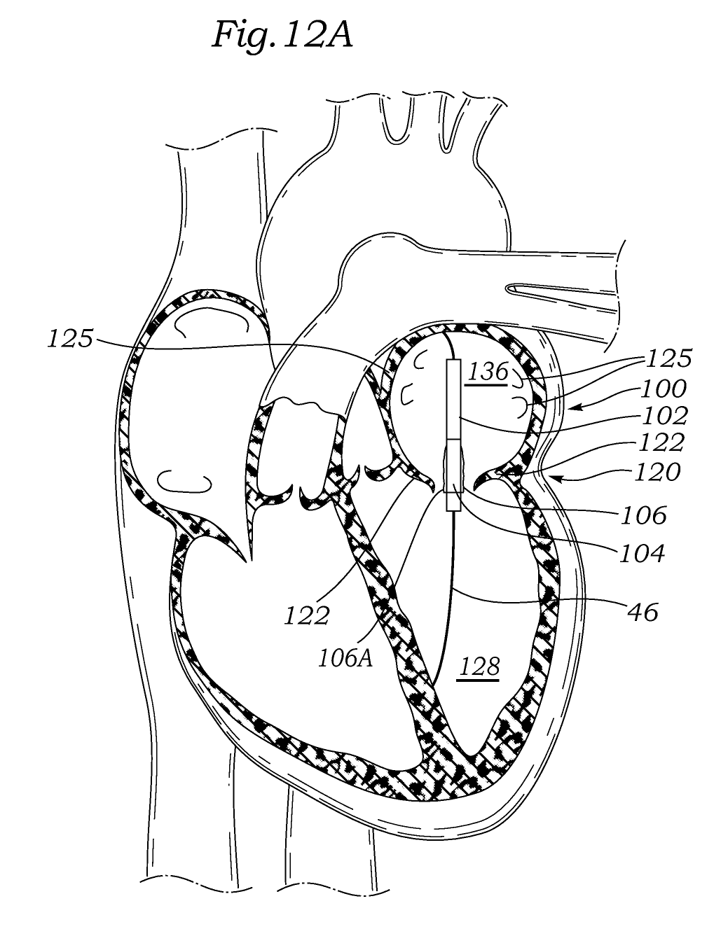

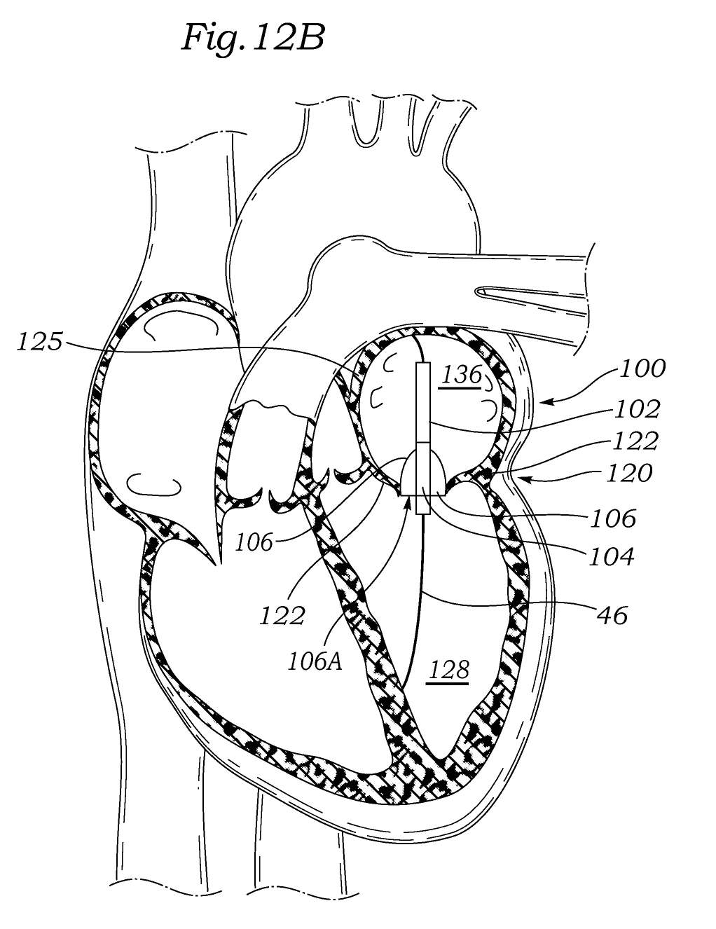

The ring 102 can be made from an elastic, shape-memory material such as Nitinol which allows the leaflet contacting member 100 to be compressed or loaded into a delivery catheter 110, then expanded to a predetermined shape within the left atrium 126, as seen in FIGS. 12A and 12B. The ring 102 can be sized to press against the walls of the left atrium 126 of the heart 124, and in some configurations within the commissure of the mitral valve 120, thereby anchoring the position of the leaflet contacting member 100, while positioning the pocket 106 at least partially through a mitral valve 120. Additionally, the lower open end 106A of the pocket 106 is positioned near or within the left ventricle 128. In this sense, the ring 102 can more generally be described as an anchoring framework or an anchoring structure.

Once positioned within the heart 124, the leaflet contacting member 100 at least partially surrounds wire 46, thereby preventing wire 46 from contacting the native leaflets 122. When leaflet contacting member 100 includes an expandable member it can also function in a manner complementary to a heart valve, opening (e.g., collapsing) during diastole to allow blood flow between the leaflets 122 and closing (e.g., expanding) during systole to restrict blood flow between the leaflets 122. As shown in FIGS. 11A and 11B, aperture 105 is spaced apart from an outer surface of the expandable member (e.g., pocket 106) so that wire 46 is prevented from contacting the leaflets 122 as the leaflets 122 move between their open and closed states.

More specifically, as blood enters the left atrium from the pulmonary veins 125 near the top of the left atrium 126, the blood flow moves downward towards the mitral valve 120. As the blood flow reaches the mitral valve 120, it pushes against the mitral valve leaflets 122 as the mitral valve 120 opens. The blood flow also pushes against a top surface of the pocket 106 of the leaflet contacting member 100, forcing out any blood that may be within the pocket 106 and causing the pocket 106 to assume a substantially deflated or compressed position, as seen in FIG. 12A. This compressed configuration of the pocket 106 provides a streamline profile that minimizes blood flow resistance and other disruptive effects that a device within the left atrium might otherwise cause. In this respect, the blood flow during diastole passes into the left atrium 126, through the mitral valve 120 and past the leaflet contacting member 100 to allow passage of the blood flow into the left ventricle 128.

During systole, backpressure from the blood in the left ventricle 128 presses against the mitral valve leaflets 122, as the papillary muscles move these leaflets 122 to a closed position. Additionally, this backpressure of blood in the left ventricle 128 enters the pocket 106 of the prosthesis 100, causing the pocket 106 to achieve an expanded shape, as seen in FIG. 12B. The mitral valve leaflets 122 coapt against the expanded pocket 106, as best seen in FIG. 11B, minimizing or even eliminating gaps that would otherwise be present between the two leaflets 122. Thus, blood flow during systole expands the leaflet contacting member 100 to reduce or eliminate any openings that would otherwise be present between the leaflets 122, ultimately reducing or preventing regurgitation of blood into the left atrium 126.

Due in part to the dynamic, flexible nature of the pocket 106, the leaflet contacting member 100 can expand to fill a wide range of opening sizes between the leaflets 122 without the need for an equally wide range of pocket sizes. In other words, the same size pocket 106 can expand to fill a relatively small opening or a relatively large opening between the mitral valve leaflets 122. Thus, the same size leaflet contacting member 100 may be appropriate for a patient with relatively severe mitral valve regurgitation as well as relatively mild mitral valve regurgitation. Different sizes of leaflet contacting member 100 may be appropriate; however, for different size mitral valves 120, since it is preferred that the pocket 106 extends along the length of the commissure of the mitral valve or the length of the “meeting line” between the two leaflets.

The leaflet contacting members are preferably delivered percutaneously by a catheter. The leaflet contacting members can be delivered to the desired heart valve using any known delivery method. For example, to deliver a leaflet contacting member to a mitral valve, the delivery catheter 110 can be fed through the femoral vein, into the right atrium and passed through a pre-made puncture in the atrial septum 125 to the left atrium. In another example, the delivery catheter 110 can be passed through the femoral artery into the aorta, through the aortic valve and into the left ventricle. Alternately, the leaflet contacting member 100 can be inserted into the left atrium 126 through an opening in the atrial wall of the heart 125 during open-heart surgery. Although the leaflet contacting member 100 can be seen and positioned more easily during open-heart procedures, percutaneous delivery is less invasive and therefore includes a substantially lower risk of complications.

One or more leads or wires (or other similar structures) that pass through one or more apertures in the leaflet contacting member can be delivered to the heart at the same time as the leaflet contacting members describe herein. Alternatively, the leaflet contacting members can be delivered to the heart before or after the delivery of the leads or wires. If the leaflet contacting member is delivered after the leads have been positioned, the leads can be rerouted through the aperture of the leaflet contacting member. If the leaflet contacting member is delivered before delivery and implantation of the leads, it may be desirable to temporarily plug or otherwise close the aperture of the leaflet contacting member. Then, when the lead is to be implanted, the plug or other aperture-blocking member can be removed and the lead can be passed through the aperture and positioned as desired within the heart.

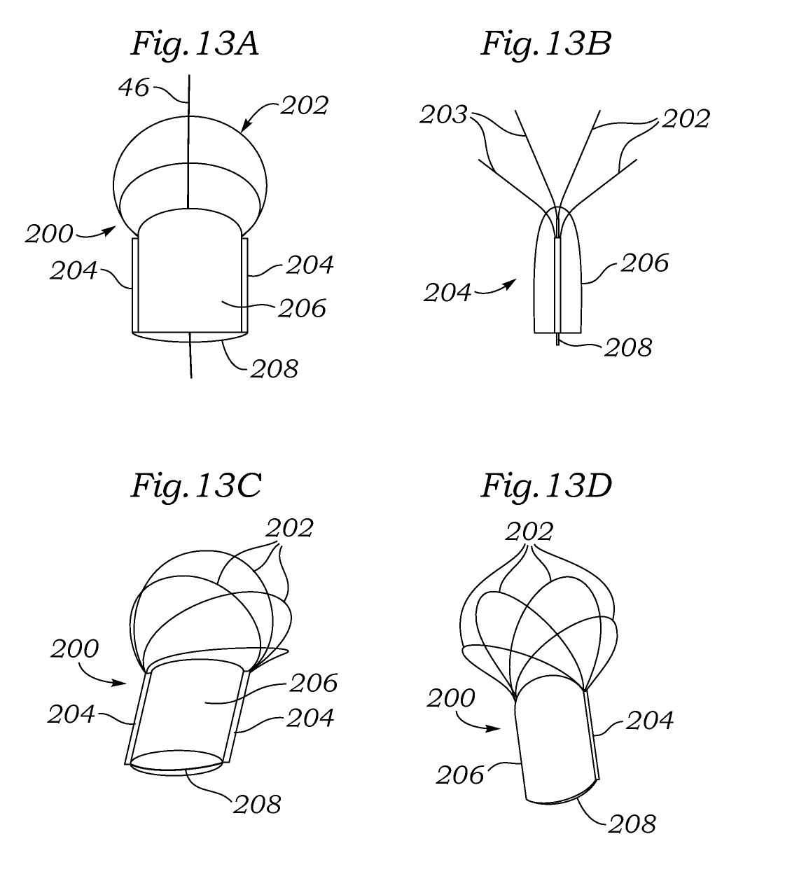

Leaflet contacting member 100 can be anchored within the heart using any appropriate anchoring structure. For example, as discussed herein with respect to the tricuspid valve, the leaflet contacting member can be anchored above or below the heart valve. Other possible anchoring mechanisms are illustrated in FIGS. 13A-13D. For convenience and clarity of the figures, wire 46 is shown passing through the leaflet contacting member 200 in FIG. 13A only.

While generally similar to leaflet contacting member 100, leaflet contacting member 200 includes an anchoring member or framework in the form of a plurality of anchoring loops 202 that expand to anchor the leaflet contacting member 200 within the left atrium 126. By securing the anchoring the leaflet contacting member 200 to the wall of the left atrium, a pocket 206 can be positioned and held between the mitral valve leaflets 122 along the length of the mitral valve commissure. In this manner, as shown in FIG. 13A, pocket 206 substantially surrounds a wire 46 that passes through an aperture of leaflet contacting member 200.

As in the other embodiments disclosed herein, leaflet contacting member 200 comprises an aperture for receiving a wire 46, so that when the leaflet contacting member 200 is positioned within a valve annulus, the wire 46 is restricted from contacting the native leaflets 122.

Pocket 206 can be supported by support arms 204 and a bottom support 208 which provide a support framework for pocket 206. Side arms 204 and bottom support 208 can be a single, unitary wire that connects to the anchoring loops 202; however multiple segments of wire can be connected together, for example by welding or soldering, as well. Support arms 204 and the bottom support 208 are preferably composed of an elastic, memory-shape material, such as Nitinol, which allows leaflet contacting member 200 to be compressed and loaded into a catheter, and then deployed to the predetermined functional size and shape. Preferably, the wires used for support arms 204 and bottom support 208 are sized and shaped to cause minimal deformation of the free edges of the leaflets 122, and therefore minimize distortion of the mitral valve geometry. In this respect, pocket support arms 204 can alternatively be described as a framework, a support structure, or a positioning frame.

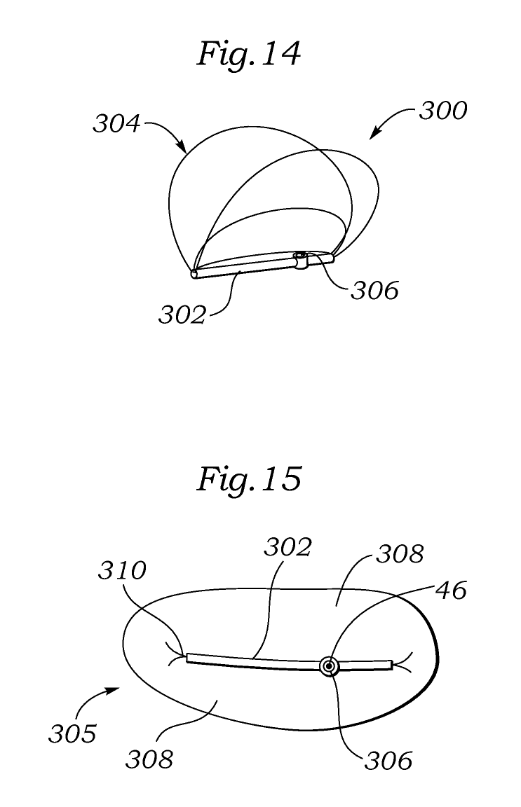

As discussed above, the leaflet contacting member can be radially collapsible and expandable to further prevent leakage from occurring within a valve. However, it should be understood that even if the leaflet contacting member need not be collapsible or expandable. For example, as shown in FIG. 14, a leaflet contacting member 300 can include a non-collapsible aperture-containing portion 302 that is capable of being positioned at or within an annulus of a valve. Preferably, aperture-containing portion 302 is anchored at or within the annulus by an anchoring member, such as anchoring member 304. The aperture-containing portion 302 has an opening or aperture 306 through which the wire 46 (or other similar structure) can pass and be substantially restricted from contacting the leaflets of the native valve. The shape of the aperture-containing portion and the location of the aperture in the aperture-containing portion can vary. Preferably, the shape and locations are determined based on the location and size of gaps that are present in the native valve leaflets as they coapt. Thus, the aperture-containing member can act to restrict leakage between the leaflets during coaptation.

FIG. 15 illustrates a view of a mitral valve 305 with leaflets 308 in a closed position. The aperture-containing member 302 can extend at least partially along a commissure 310 of the valve so that as the leaflets 306 coapt, the leaflets 308 contact an outer surface of the aperture-containing portion 302 of the leaflet contacting member 300. One or more wires (leads) 46 can be passed through the aperture 306 and the outer surface of the aperture-containing member can substantially prevent lead 46 from contacting the leaflets 308 during coaptation.

As described above, leaflet contacting members can have expandable members to further reduce leaks between valve leaflets during coaptation. Described below is a diagnostic tool for use in determining whether the use of such a leaflet contacting member (with or without an aperture for receiving a lead) would be effective with a particular patient to reduce leakage and/or regurgitation.

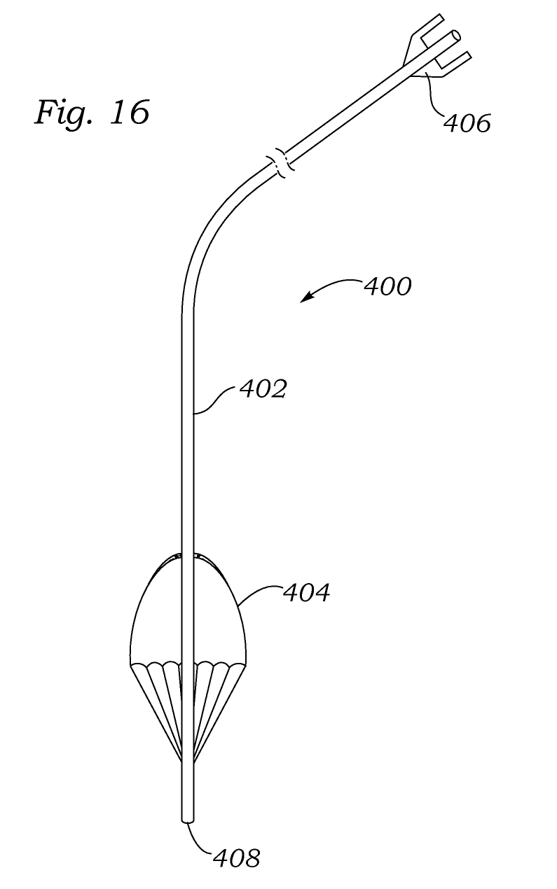

Referring to FIG. 16, an apparatus 400 includes an elongate member 402 (e.g., a catheter) and a temporary leaflet contacting member 404 at or near a distal end of the apparatus. For example, the temporary leaflet contacting member 404 can be coupled to a distal end of the elongate member 402. The temporary leaflet contacting member 404 is preferably substantially similar (in shape and function) to the leaflet contacting member for which the physician would like to determine a potential effectiveness. Thus, for example, if a physician is considering implanting a leaflet contacting member 100 (as described herein), temporary leaflet contacting member 404 should be substantially similar to leaflet contacting member 100 in size, shape, and function. The primary difference between the temporary leaflet contacting member 404 and the more permanent leaflet contacting member (e.g., leaflet contacting member 100 in this example) is that temporary leaflet contacting member 404 is designed as a diagnostic tool that is intended for temporary placement within the body. Thus, the temporary leaflet contacting member 404 is configured to be removed (retracted) from the body. As such, temporary leaflet contacting member 404 preferably is not connected to an anchoring member and is, instead, coupled to a delivery system that is removable from the patient's vasculature.

Temporary leaflet contacting member 404 can be delivered to the desired heart valve via the elongate member 402 using any known delivery method, such as those methods discussed above for the leaflet contacting members. Thus, for example, temporary leaflet contacting member 404 of apparatus 400 can be delivered surgically through an opening the chest or delivered percutaneously to the treatment site. If the delivery is performed percutaneously, elongate member 402 can comprise a catheter that extends through the vasculature of a patient to the treatment site in the heart.

Once the temporary leaflet contacting member 404 is delivered to the heart, the temporary leaflet contacting member 404 can be positioned at or within the annulus of the heart valve that is under consideration for treatment with a more permanent leaflet contacting member. For example, if the valve to be treated is the mitral valve, temporary leaflet contacting member 402 is positioned between the leaflets of the mitral valve.

The temporary leaflet contacting member 404 can be moved around within the mitral valve to different positions to determine where leakage between the leaflets is occurring or at least where the leakage is most problematic, such as by moving the catheter 402, which can extend outside of the body. In addition, at each position within the mitral valve, the physician can determine whether a leaflet contacting member of the same size, shape, and/or function as the temporary leaflet contacting member 404 would be effective to reduce the occurrence of regurgitation in the valve. If the temporary leaflet contacting member 404 does not appear to be effective, the physician can make a determination as to whether a leaflet contacting member of a different size, shape, or function might be effective based on the effectiveness or ineffectiveness of the temporary leaflet contacting member 404.

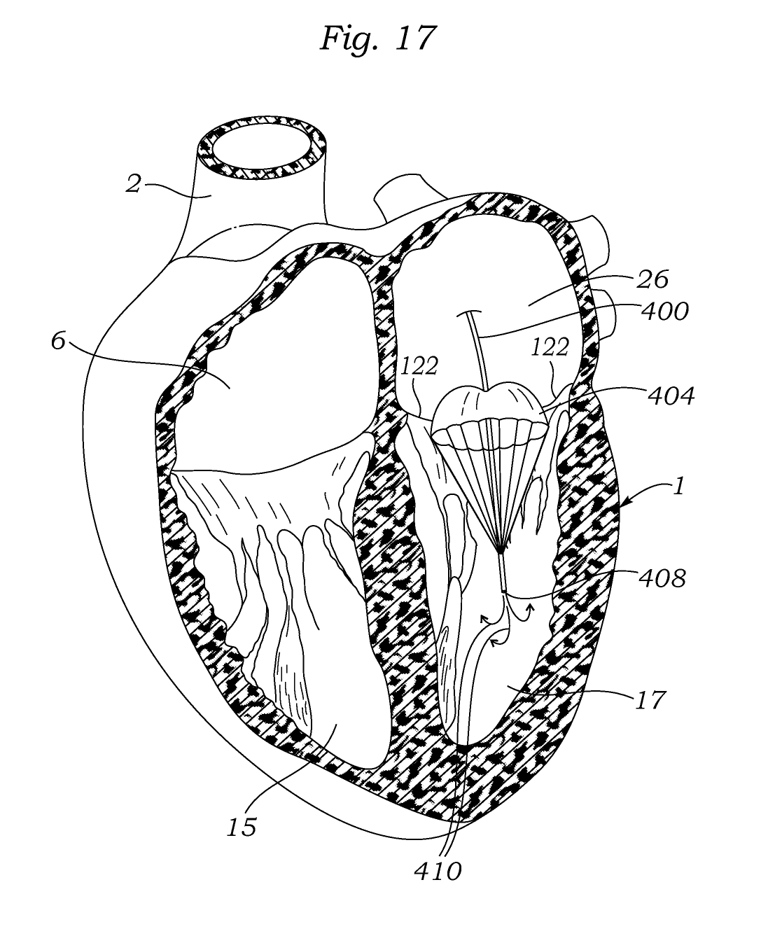

Catheter 402 preferably includes a lumen for the delivery of a fluid 410 into a heart chamber downstream of the leaflet contacting member (e.g., into the left ventricle). As shown in FIG. 17, while the temporary leaflet contacting member 404 is positioned between the mitral valve leaflets 122, fluid 410 can be delivered through the catheter 402 and into the left ventricle. Fluid 410 can be delivered into apparatus 400 through a luer connection 406 (or other similar connection) located outside of the vasculature of the patient. Fluid 410 can be pumped through the lumen of the elongate member 402, past the temporary leaflet contacting member 404 (or at least past a point where the temporary leaflet contacting member is attached or coupled to the elongate member 402), and out the distal end (opening) 408 of the elongate member 402. The distal end 408 is preferably positioned within the left ventricle so that fluid is delivered into the left ventricle.

The delivery of fluid into the left ventricle increases the fluid pressure in the left ventricle which allows the physician to more easily identify leaks (regurgitation) between the mitral valve leaflets and the temporary leaflet contacting member. Thus, apparatus 400 allows the physician to make a more accurate determination as to whether the patient would be benefit from a heart valve treatment that uses a leaflet contacting member that is similar or substantially identical to the temporary leaflet contacting member.

In order to determine the effectiveness of the temporary leaflet contacting member (and therefore the effectiveness of a similar permanent leaflet contacting member), the physician can observe the effectiveness of the temporary leaflet contacting member in restricting or preventing regurgitation between the leaflets. In addition, as discussed above, the physician can repeatedly move or reposition the temporary leaflet contacting member between the leaflets to make multiple determinations of the effectiveness of the temporary leaflet contacting member, with each determination being based on a different position of the temporary leaflet contacting member.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as our invention all that comes within the scope and spirit of these claims.

We claim:

1. A prosthesis for positioning within an annulus of a heart valve, the prosthesis comprising:
a leaflet contacting member having an outer surface, an inflow side, an outflow side, and an aperture that extends through at least a portion of the leaflet contacting member, the aperture being sized to allow one or more wires to extend through the leaflet contacting member from the inflow side to the outflow side; and
an anchoring member coupled to the leaflet contacting member, the anchoring member configured to anchor the leaflet contacting member within the annulus of a heart valve,
wherein the outer surface is configured to contact one or more leaflets of the heart valve during coaptation of the leaflets, and the contact of the leaflets with the outer surface prevents the leaflets from contacting the one or more wires when positioned to extend through the aperture of the leaflet contacting member.

2. The prosthesis of claim 1, wherein the aperture has a width that is substantially the same as the width of the one or more wires that extend through the aperture, thereby substantially restricting blood flow through the aperture when the one or more wires extend through the aperture.

3. The prosthesis of claim 1, further comprising:
a plug member configured to restrict blood flow through the aperture when the one or more wires extend through the aperture.

4. The prosthesis of claim 1, wherein the anchoring member has a compressed state sized to fit within a delivery catheter and an expanded state sized for fixation on at least a portion of a wall of a left atrium.

5. The prosthesis of claim 4, wherein the anchoring member comprises a plurality of loops formed of a shape memory material.

6. The prosthesis of claim 1, wherein the anchoring structure extends into the right ventricle and is secured to a wall of the right ventricle.

7. The prosthesis of claim 1, wherein the anchoring structure comprises an electrode tip coupled to a distal end of the one or more wires.

8. The prosthesis of claim 1, wherein the leaflet contacting member comprises an expandable member, the expandable member having an expanded state that restricts blood flow between the leaflets and the leaflet contacting member and a contracted state that allows blood to flow between the leaflets and the leaflet contacting member.

9. The prosthesis of claim 1, wherein the leaflet contacting member has a length that is substantially equal to a length of a commissure of the leaflets.

10. The prosthesis of claim 9, wherein the aperture is positioned at a substantially central location along the length of the leaflet contacting member.

11. The prosthesis of claim 9, wherein the aperture is positioned off center relative to the length of the leaflet contacting member.

12. A system for preventing contact between a wire and one or more valve leaflets, the system comprising:
a prosthetic device comprising a main body that is sized for placement at least partially between two valve leaflets that are movable between an open state and a closed state, the main body comprising an opening that extends from a first side of the main body to a second side of the main body; and
at least one wire configured to extend through the opening of the main body from the first side to the second side,
wherein the opening is spaced apart from an outer perimeter of the main body so that, when the prosthetic device is placed between the two valve leaflets, the wire does not contact either of the two valve leaflets when the valve leaflets are in the open or closed states.

13. The system of claim 12, wherein the prosthetic device further comprises an anchoring member, the anchoring member being configured to securely hold the main body at least partially between the two valve leaflets.

14. The system of claim 13, wherein the anchoring member has a compressed state sized to fit within a delivery catheter and an expanded state sized for fixation on at least a portion of a wall of a left atrium.

15. The system of claim 14, wherein the anchoring member comprises a plurality of loops formed of a shape memory material.

16. The system of claim 12, wherein the opening is substantially the same size as the one or more wires that extend through the opening, thereby substantially restricting blood flow through the opening when the one or more wires extend through the opening.

17. The system of claim 12, wherein the main body further comprises an expandable member having an outer surface that surrounds the aperture, the expandable member having an expanded state that restricts blood flow between the leaflets and the outer surface of the expandable member and a contracted state that allows blood to flow between the leaflets and the outer surface of the leaflet contacting member.

18. The system of claim 12, wherein the main body has a length that is substantially equal to a length of a commissure of the leaflets.

19. A method for delivering a wire through a valve of the heart, the method comprising:
providing a leaflet contacting member having an outer surface and an aperture passing through the leaflet contacting member;
positioning the leaflet contacting member at least partially between two leaflets of a valve so that the outer surface of the leaflet contacting member contacts the leaflets when the leaflets undergo coaptation; and
passing one or more wires through the aperture of the leaflet contact member, the aperture being positioned such that the one or more wires do not contact the leaflets.

20. The method of claim 19, further comprising:
providing an anchoring member coupled to the prosthetic device; and
anchoring the leaflet contacting member at least partially between the two leaflets.

21. The method of claim 19, further comprising:
providing a plug member; and
positioning the plug member at or around the aperture to restrict blood flow through the aperture.

22. A diagnostic tool comprising:
an elongate member having a proximal portion, a distal portion, and a distal opening, the elongate member having a lumen that extends from the proximal portion to the distal portion;
a temporary coaptation member coupled to the distal portion of the elongate member at an attachment portion, the temporary coaptation member being configured to be positioned between two leaflets,
wherein the attachment portion is located proximally to the distal opening in the elongate member, the distal opening being in fluid connection with the lumen.

23. The tool of claim 22, further comprising a fluid delivery device coupled to the proximal portion of the diagnostic tool and configured to pump fluid from the proximal portion to the distal portion.

24. The tool of claim 22, wherein the temporary coaptation member comprises an expandable member that has an expanded state that restricts blood flow between the leaflets and the expandable member and a contracted state that allows blood to flow between the leaflets and the expandable member.

25. A method of using a diagnostic tool for determining an effectiveness of an temporary coaptation member to minimize regurgitation in a heart valve in a body of a patient; the method comprising:
providing an elongate member having a proximal portion and a distal portion;
providing an temporary coaptation member coupled to the distal portion of the elongate member;
positioning the temporary coaptation member at least partially between two leaflets of heart valve, the leaflets being movable between an open state and a closed state;
monitoring the effectiveness of the temporary coaptation member in restricting blood flow between the leaflets and the temporary coaptation member when the leaflets are in the closed state; and
removing the expandable member from the patient's body.

26. The method of claim 25, further comprising:
providing a lumen that extends through the elongate member from the proximal portion to the distal portion, the lumen being in fluid connection with a distal opening in the elongate member;
positioning the distal opening of the elongate member in the left ventricle; and
delivering fluid through the lumen into the left ventricle to increase a fluid pressure in the left ventricle.

27. The method of claim 25, further comprising:
moving the temporary coaptation member to a different position at least partially between the leaflets; and
monitoring the effectiveness of the temporary coaptation member in restricting blood flow between the leaflets and the temporary coaptation member when the leaflets are in the closed state.

28. The method of claim 25, wherein the temporary coaptation member comprises an expandable member that has an expanded state that restricts blood flow between the leaflets and the expandable member and a contracted state that allows blood to flow between the leaflets and the expandable member.
